(19) United States
(12) Patent Application Publication
(10) Pub. No.: US 2025/0281689 A1
(43) Pub. Date: Sep. 11, 2025

(54) IMPROVED INJECTION SYSTEM FOR DRUG ADMINISTRATION DEVICE

(71) Applicant: Société industrielle de Sonceboz S.A., Sonceboz-Sombeval (CH)

(72) Inventors: Thierry Martin, Mont de Vougney (FR); Jocelyn Staub, Charquemont (FR)

(21) Appl. No.: 18/859,301

(22) PCT Filed: Apr. 26, 2023
(86) PCT No.: PCT/EP2023/060981

§ 371 Date: Oct. 23, 2024

(30) Foreign Application Priority Data
Apr. 26, 2022 (FR) 2203901

(51) Int. Cl. A61M 5/142; A61M 5/168

(52) U.S. Cl. CPC A61M 5/14248; A61M 5/168; A61M 2005/14252; A61M 2202/0007; A61M 2205/10; A61M 2205/8281

(57) ABSTRACT

A device for transdermal drug administration comprises a housing, a lower casing of the housing defining a cutaneous connection surface having an opening for the passage of a transdermal injection canula, a reservoir containing an active ingredient intended to be administered, a pump system comprising a pump body, a valve piston and/or a pump piston that can be moved in the pump body by at least one drive system, a transdermal injection system and an electronic control system. The injection system is controlled by a translational element interacting with a connection means inserted between the pump piston or the motorized valve piston on one side and the injection system on the other side, the connection means being movable between a first position that it occupies until the injection system is activated and a second, irreversible retracted position into which it is switched by the triggering of the injection system.

### CROSS-REFERENCE TO RELATED APPLICATIONS

This application is a national phase entry under 35 U.S.C. § 371 of International Patent Application PCT/EP2023/060981, filed Apr. 26, 2023, designating the United States of America and published as International Patent Publication WO 2023/209020 A1 on Nov. 2, 2023, which claims the benefit under Article 8 of the Patent Cooperation Treaty of French Patent Application Serial No. FR2203901, filed Apr. 26, 2022.

### TECHNICAL FIELD

The present disclosure relates to the field of external drug administration systems, in particular, transdermal drug administration systems, incorporating a pump system and a transdermal administration system. Typically, the system is integrated into a housing whose interior maintains the sterility of the fluidic, self-contained part, which adheres to the patient's abdomen or chest or body part, and delivers the substance to the patient via a canula that is inserted into the patient subcutaneously.

### BACKGROUND

The medical treatment of many diseases requires continuous infusion of medication, via subcutaneous and intravenous injections, particularly for drugs containing large molecules that cannot be digested when administered orally, such as insulin, biologics and biosimilars.

Patients suffering from chronic diseases such as of the immuno-oncology type, or requiring recurrent post-operative treatment, need bolus injections of medication, usually administered by nursing staff. The quantity of these injections can vary from a few milliliters to several tens of milliliters. As the flow rate is capped to be tolerated by the patient, the injection time increases proportionally with the volume injected, and has a direct impact on the workload of nursing staff. Automatic drug injection pumps have been developed to free up nursing staff time when administration via the pump must be supervised by trained personnel, and to enable patients to self-administer drugs safely thanks to a simplified automatic system, giving them routine autonomy and drastically reducing the frequency with which they need to visit the hospital.

Basal and bolus volumes must be administered in precise doses according to individual prescriptions. As a result, drug injection pumps must be highly reliable, ensuring that patients and caregivers can monitor the correct dose.

These pumps are also used for the injection of biological drugs, or active anticancer ingredients, requiring shorter injection times, from a few tens of minutes to a few hours, at less frequent weekly or monthly intervals.

To avoid problems of re-sterilization of ambulatory devices intended for home treatment, single-use devices are preferred. The simplicity of the device's components is therefore essential to ensure a sale price that is not prohibitive.

Usually, the patient fills the reservoir with the active ingredient, for example, from a vial, attaches the needle and administration tube to the reservoir outlet, then inserts the reservoir into the pump housing. After purging the air from the reservoir, tube and needle, the patient inserts the needle assembly, penetration element and canula into a selected location on their body, and removes the penetration element. To avoid irritation and infection, the subcutaneous canula should be replaced and discarded with the empty reservoir.

Alternatively, the patient or caregiver may use an additional transfer station to automatically transfer the contents of the vial into the medical device's internal reservoir. To do this, the user generally needs to connect the vial (and vial adapter if not already integrated into the transfer station) on one side, and the medical device on the other, to the transfer station, then start the transfer sequence.

These devices are equipped with an internal energy source that powers the inner workings when the drug is injected into the patient. As these devices can be stored for long periods, it's crucial to preserve the power source during this time, and to power on the system only when required.

Known from the prior art is the patent application US2019365993 describing a drug administration device comprising a transdermal administration system having a needle actuation mechanism configured for transdermal insertion of a canula and a needle guide element for guiding axial movement of a needle and canula. The needle actuation mechanism comprises a cam element rotatable relative to the needle guide element. The guide element guides a needle attached to a needle holder and a canula attached to a canula holder. The needle and canula holders each comprise an engagement portion, wherein the engagement portion of the needle holder is configured to engage with first and second cam surfaces and the engagement portion of the canula holder is configured to engage with a locking surface.

Also known is patent application US20200108201A1 describing a therapeutic substance administration device comprising a fluid path having a reservoir needle, a body needle, a body needle injection mechanism and a pumping assembly (a) configured to pump substance from the reservoir to the subject, (b) shaped to define a pump chamber, and (c) comprising a plunger disposed within the pump chamber. The plunger moves back and forth through a plurality of discrete motion phases. A first motion phase of the plunger actuates a first operation from a group of operations comprising (a) driving the reservoir needle to penetrate the reservoir, (b) advancing the body needle into the body of the subject, (c) withdrawing the substance from the reservoir, (d) pumping the substance into the subject, and (e) retracting the body needle. A second motion phase of the plunger actuates a second operation from the group of operations.

Patent application US20210330893A1 describes a manual auto-injector for drug administration. The auto injector comprises a housing, a syringe holder configured to receive a syringe; a first drive module adapted to move the syringe holder relatively to the housing; and a rigid needle shield remover comprising a first part adapted for separating a rigid needle shield from the syringe.

In the solution described in patent EP3354303, although the device features two electromagnetic actuators, it is not possible to control the needle and canula actuation mechanism independently of the operation of the pump. The mechanism is actuated by a pre-stressed spring, and is locked in position by the rod of the pump's piston before first use. When the pump is used for the first time, the rod releases the lock on the needle and canula actuating mechanism, which cannot then be returned to the standby position.

The solution proposed by patent application US20210330893A1 is not entirely satisfactory. The same motorized element actuates the pump and the injection needle triggering system, in a succession of movements with increasing amplitudes, but always limited by the activation position of the first and then the second triggering of the ejection mechanism. As a result, this solution does not make full use of the motorized stroke, which is limited by the position of the first trigger for needle extension and the second trigger for needle retraction. The stroke that can be used for pumping, during the drug injection phase via the needle inserted into the patient's body, is therefore reduced and limited by the needle movement mechanism, and thus requires a greater number of cycles, which increases the motor's power consumption; unless the volume of the pump body is increased, which is however unfavorable as it forces the device to take up more space.

Moreover, to make the most of the available stroke, it is necessary to control the movement with great precision, as any error would result in the unexpected triggering of the needle extension and retraction mechanism.

### BRIEF SUMMARY

In order to remedy the drawbacks of the prior art, the present disclosure relates, in its most general sense, to a transdermal drug administration device.

The system comprises:
a housing, a lower casing of the housing defining a cutaneous connection surface with an opening for the passage of a transdermal injection canula,
a reservoir containing an active ingredient intended to be administered,
a pump system comprising a pump body, a valve piston and/or a pump piston that can be axially moved in the pump body by at least one drive system, and
a transdermal injection system and an electronic control system for operating the drive system(s) of the pump system and for releasing the transdermal injection system,
   wherein the injection system is controlled by a translational element interacting with a connection means inserted between the pump piston or the motorized valve piston on one side, and the injection system on the other side, the connection means being movable between a first position, which it occupies until the injection system is activated and a second, irreversible position, wherein it is switched by the triggering of the injection system, and wherein second position it is retracted so as to no longer present any zone of possible interaction with the piston, over the whole stroke of the piston.

In addition, the electronic system can control:
over a limited part of the nominal axial stroke of the pump piston or the valve piston, the pumping function or the valve function only, and
over at least an additional part of the stroke, the axial displacement of the pump piston or the valve piston so as to drive an additional function independent of the pumping function or the valve function, the additional function not being activated during the axial displacement of the pump piston or the valve piston over the nominal stroke.

In particular, the additional function can be the triggering of the transdermal injection system.

In this case, the transdermal injection system may comprise:
a mechanism for actuating an assembly comprising a needle connected to a needle holder, the canula connected to a canula holder, and a guide element configured to guide the axial displacement of the needle and canula,
   the actuating mechanism comprising:
a cam element rotatable relative to the needle guide element, the needle holder and canula holder each comprising a protuberance cooperating with a rib of the guide element,
a pre-loaded spring for imparting rotational movement to the cam element relative to the needle guide element, for sequentially controlling needle insertion and needle retraction movement such that the needle and canula are moved from a retracted position to an extended position upon rotation of the cam element through a first predetermined angle, by cooperation of the protuberances of the needle holder and canula holder with the rib of the cam element, and such that the needle is returned to the retracted position upon further rotation of the cam element through a second predetermined angle by cooperation of the protuberance of the needle holder with the rib of the cam element, whereby during rotation through the second predetermined angle, the protuberance of the canula holder is maintained in the extended position by cooperation with the cam element.

In one variant, the injection system is provided with a locking device bearing against a wall of the cam element so as to prevent the cam element from rotating due to the action of the pre-loaded spring.

In particular, the locking device is provided with a connection means rotatable about an axis and a translational element translatable in a plane orthogonal to the axis, a distal end of the translational element being in contact with the wall of the cam element, the proximal end resting on a surface of the connection means, an opposite surface of the connection means resting against a surface of a protuberance of the lower casing impeding any translational movement of the translational element, and in that the end of the rod of one of the pistons is movable through the additional stroke to a position where it comes into contact with the connection means to impart a rotational movement thereto, the rotational movement causing the release of the locking device by releasing the contact between the proximal end of the translational element and the surface of the connection means.

In another variant, the additional function is the triggering of a system for perforating a membrane separating the recess for a cartridge containing an active ingredient from the interior space of the housing.

“Independent” means that one function does not necessarily drive the other in a systematic, coordinated way. The rod can control:
the pumping alone, excluding the additional function, during the “nominal stroke,” and
the triggering of the additional function in the complementary part of the stroke only.
   In this additional part of the stroke, the rod can also control the pumping, or not, or only over a limited part of the additional stroke.

### BRIEF DESCRIPTION OF THE DRAWINGS

For a better understanding of the invention, and to show how embodiments thereof may be implemented, reference will now be made, by way of example, to the appended drawings wherein:

FIG. 1 shows a top view of the locking device of the insertion mechanism in its initial state,

FIG. 2 shows a top view of the locking device of the insertion mechanism in its final state,

FIG. 3 shows the needle insertion mechanism alone in perspective view and partial cross-section,

FIG. 4 shows a perspective view of the locking mechanism associated with the pump system in partial cross-section and housed in the lower casing of the housing,

FIG. 5 shows a side view of the pump system associated with the locking mechanism,

FIG. 6 shows a perspective view of the complete fluidic system integrated in the lower casing of the housing,

FIG. 7 shows a side cross-section of the pump,

FIG. 8 shows a top view of the pump body, from which the valve piston emerges,

FIG. 9 shows a front cross-sectional view of the pump,

FIGS. 10A-10D show two pump hole shapes for connecting tubing and the fluid flow cross-sections obtained for different valve piston positions,

FIGS. 11-13 show a perspective view of the fluidic system compatible with an external vial for various steps of vial insertion, with FIG. 11 showing all the elements before assembly, FIG. 12 showing the vial positioned in the adapter and FIG. 13 showing the vial and adapter assembly assembled to the dock and in final position,

FIG. 14 shows a cross-sectional side view of the adapter and dock,

FIG. 15 shows a side view of the dock,

FIGS. 16 and 17 show top views of the dock integrated into the lower casing in the initial and final positions of the activation stroke, respectively,

FIGS. 18 and 19 show cross-sectional top views of the dock integrated into the lower casing in the initial and final positions of the activation stroke, respectively.

### DETAILED DESCRIPTION

### General Principle of Triggering the Injection System

Firstly, a preferred embodiment of the present disclosure concerns a solution using a canula and a needle. The injection mechanism then operates in multiple steps:

Inserting the needle surrounded by the canula,
Removing the needle alone, with the canula remaining implanted in the patient's body, as soon as the canula-needle assembly has reached its position in the patient's body,
Transferring the drug through the canula, with the needle locked in the withdrawn position and the canula locked in the insertion position, for the duration of the treatment, and
After treatment, removing the canula by taking off the device.

However, the present disclosure is not limited to this preferred embodiment, and can also provide for a more rudimentary mechanism as proposed by patent application US20210330893A1, consisting of simply moving a hollow needle that remains implanted in the patient's body.

The present disclosure is based on the interposition of a connection means (105), inserted between the motorized pump piston (205) or valve piston (202) on the one hand, and the injection system (100) on the other.

This connection means (105) is movable between a first position, which it occupies until the needle or needle-canula assembly is commanded to extend, and a second, irreversible position, wherein it is switched by triggering of the injection system (100), and wherein it is held definitively and irreversibly.

In the second position, this connection means (105) is eliminated so that there is no longer any possible area of interaction with the piston (202, 205), over the whole stroke of the piston. The stroke comprises a nominal stroke and a release stroke. In contrast to the solution proposed by US20210330893A1, where a zone of interaction must remain in order to trigger the retraction of the needle once the connection means (105) has been removed, the whole stroke of the piston (202, 205) can be used for the purposes of valve operation or pumping.

In the example described showing an embodiment of this connection means (105), it takes the form of an element rotatable about an axis (154). In its initial position, it blocks the translational element (104), which locks the injection system (100). When the piston comes into contact with the connection means (105), it causes it to tilt about the axis (154), releasing the translational element that frees the operation of the injection system (100), and moving the connection means (105) into its second position where it is held permanently. This is ensured, for example, by a rib (171, 172) that leads this part into its second position, where it is held by a resilient return means. In this second position, the connection means (105) is definitively removed to allow the piston to move through its whole stroke, to ensure pumping, without any further direct or indirect interaction with the injection system (100).

It should be noted that the present disclosure is not limited to a drug administration device implementing this triggering mode of the injection system, and includes other inventive technical choices that are associated in one exemplary embodiment with this triggering mode, but can also be utilized without this triggering mode.

### Detailed Description of Multifunction Operation and Release of the Insertion Mechanism as Controlled by the Piston Rod

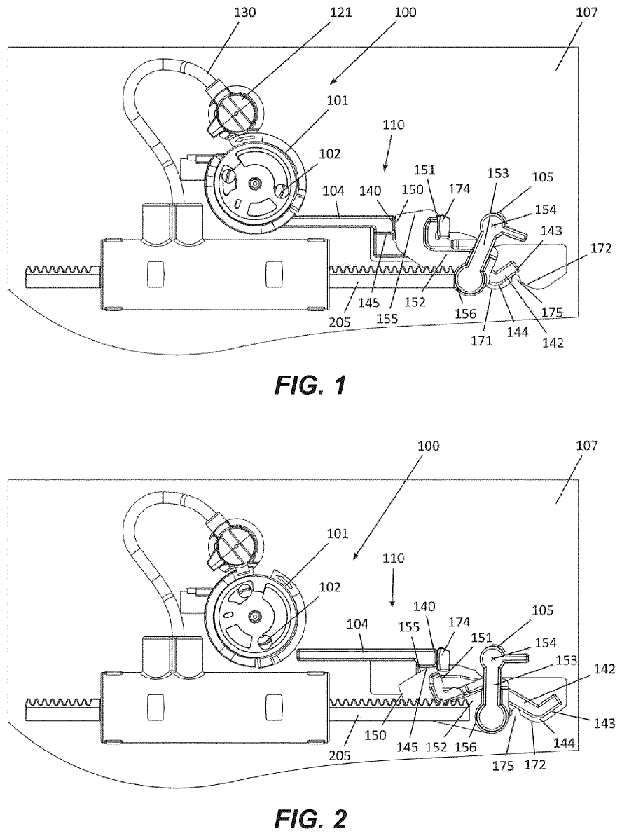

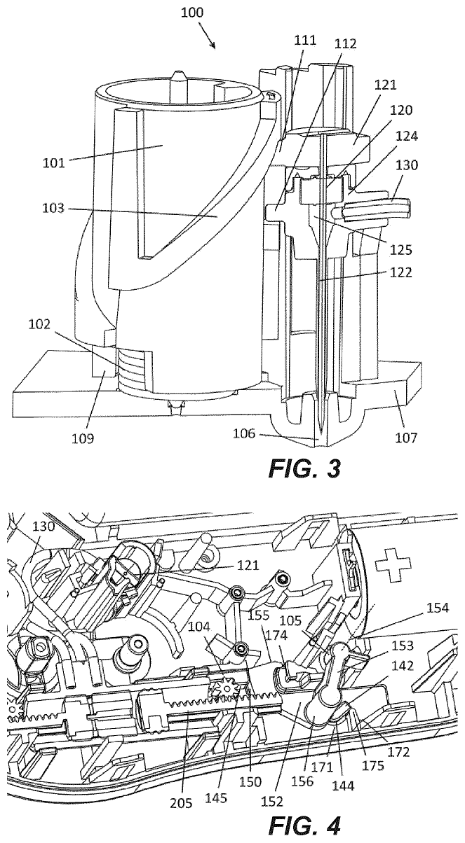

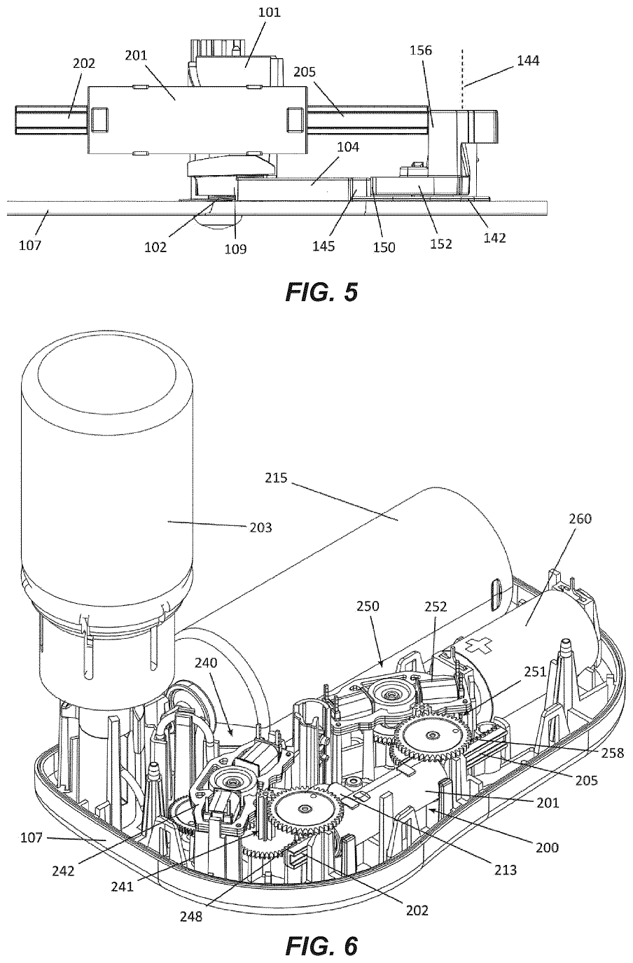

FIGS. 1-5 show a first embodiment of the locking device of the needle insertion mechanism according to the present disclosure, with FIG. 1 representing the needle insertion mechanism alone, as described in international patent application WO2018141697A1. FIGS. 2 and 3 show the needle locking and insertion mechanism in a top view, with FIG. 2 representing the device in the initial state, known as “armed,” and FIG. 3 showing the mechanism after release and needle insertion. FIG. 4 shows the needle locking and insertion mechanism in a perspective view and FIG. 5 shows the needle locking and insertion mechanism, without the cam element, in a side view.

The injection system (100) comprises, in particular, a needle insertion mechanism consisting of a cam element (101), and shown in more detail in FIG. 1 with a partial cross-sectional view. The cam element (101) has a rib (103) whose advantageous profile cooperates with protuberances (111, 112) of a needle holder (121) and a canula holder (124) enabling the insertion of the needle (120) and the flexible canula (122), then the withdrawal of the needle (120) alone, in a rotational movement of the cam element (101) by less than 360°. The canula holder (124) has a conduit for receiving the tube (130) carrying the fluid to be injected through the canula (122), this conduit being connected to the canula (122) by an internal cavity (208). The insertion of the needle (120) and canula (122) is guided through a through-hole (106) in the housing (107). The rotational movement of the cam element (101) is induced by a spiral spring (102), preloaded in the initial state, the cam element (101) being held in position by a locking system (110) described more particularly in FIGS. 2-5. The insertion sequence of the needle (120) is triggered by a release of the locking system (110), allowing free rotation of the cam element (101) and therefore insertion of the needle (120) thanks to the discharging of the spring (102). It should be noted that this sequence of movements is irreversible, and that when it is completed, the spring (102) is in the unloaded state and opposes movement of the cam (101) in the opposite direction to that used for insertion of the needle (120) and canula (122). In this way, once the sequence has been completed, the needle (120) is retracted back into the medical device and cannot be deployed again, leaving the device in a state that avoids any risk of injury to the user. The use of a retractable needle (120) housed within a flexible canula (122) does not limit the present disclosure and is especially useful for guaranteeing the sterility of the fluid conduit as long as the needle is located within the canula. The person skilled in the art could easily imagine other alternatives for creating the fluid interface between the device and the patient.

More specifically, the locking device comprises a translational element (104) and a connection means (105), with a degree of freedom in rotation, both guided by the lower casing (107) of the housing in respective translational and rotational movements.

The translational element (104) has an arm (140), the distal end of which rests on a wall (109) of the cam element (101), more visible in FIG. 5, and the proximal end (140) rests on a surface (150) of the connection means (105), and an elastic protrusion (142) ensuring that the locking device (110) is held in its final position after release, by cooperation with the groove (172) of the lower casing (107) of the housing. The connection means (105) has a surface (151) opposite the surface (150), this opposite surface (151) resting on a protuberance of the lower casing (107) of the housing. Thus, in the locked position, the cooperation of the translational element (104) and the connection means (105) enables the entire preload force of the spiral spring (102), exerted on the cam (101), to be transmitted directly to the lower casing (107) of the housing, thus preventing any possible movement. This also ensures a longer system life during storage, while the use of direct load transfer from the housing prevents the risk of the parts deforming over time.

The locking device (110) can be released by moving the connection means (105). The connection means (105) has a body (152) provided with a lever arm (153) for guiding the body (152) in rotation about the axis (154). The rotation of the connection means (105) causes the surface (150) and the opposite surface (151) to slide on the opposing surfaces (141, 174) of the translational element (104) and lower casing (107), respectively.

When the rotation of the connection means (105) is sufficient for the surface (150) to lose contact with the surface (141) of the translational element (104), the locking mechanism (110) is released and the translational element (104) performs a translational movement induced by the pre-loading force of the spiral spring (102). During the translational movement of the translational element (104), the curved profile (143) of the elastic protrusion (142) slides along the grooves (171, 172) of the housing (107), causing the end of the elastic protrusion to move orthogonally to the translational movement of the translational element. When the apex (144) of the curved profile (143) passes the separation (175) between the two grooves (171, 172), the translational element (104) is no longer in contact with the cam (101), but the translation movement continues along the groove (172) thanks to the release of the elastic energy accumulated by the elastic protrusion (142) as it moves along the first groove (171), finally arriving at the stable equilibrium position shown in FIG. 3. During the translational movement of the translational element (104), the lower surface (145) of the translational element (104) slides over an upper surface (155) of the connection means (105) so as to force the connection means (105) to move into abutment with a protrusion (173) on the lower casing (107) of the housing. The complementary profile of the surfaces (145, 155) advantageously allows the translational element (104) to continue translating following this moving into abutment. while at the same time blocking the rotational movement opposite the abutment in the final position as shown in FIG. 3.

In this way, once the locking device has been released, the elastic protrusion ensures a safe end position wherein the translational element (104) and the connection means (105) are immobilized, even in the event of sudden movement or impact on the medical device.

Before the locking device is released, the translational element (104) and the connection means (105) are held in position by the friction of the surface (150) and the opposite surface (151) on the surfaces (141, 174), respectively. The rotation of the connection means (105), and hence release of the locking device, is achieved by pressing the end of the pump piston (205) against a circular surface (156) of the lever arm (153). In the embodiment shown, this support is obtained on an extreme part of the displacement stroke of the pump piston (205), advantageously enabling the pumping function to be performed before the locking device is released, enabling, for example, the injection fluid to be transferred from an external reservoir to an internal reservoir before the device is placed on the patient. During this operation, the piston stroke of the pump can be easily limited by software.

Of course, once the locking device (110) has been triggered by a first movement of the pump piston (205) on the end part of its stroke, the pump piston (205) can benefit from the full stroke for the pumping function and thus increase, for each pump cycle, the volume transferred from the internal reservoir to the patient.

Note that the pump piston (205) only ensures the rotational movement of the connection means (105) until the locking mechanism (110) is released, as described above. In order to ensure free movement of the piston over this end stroke, it is necessary, after release of the locking device (110), to maintain the connection means (105) in a safety position as permitted here by its cooperation with the translational element (104).

### Detailed Description of the Pump Body With Oblong Holes

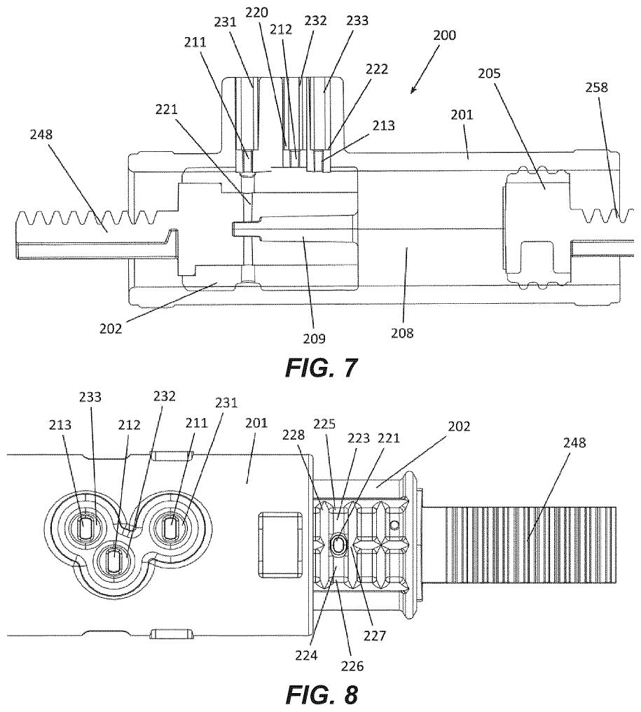

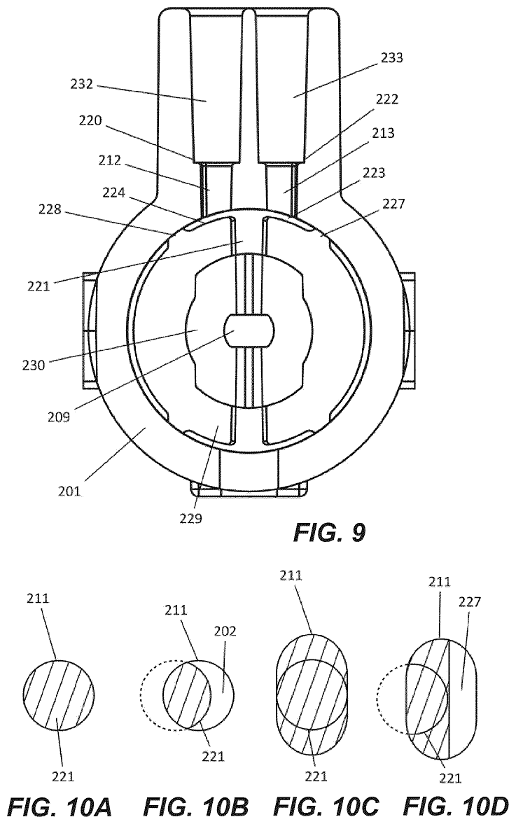

One embodiment of the pump system (200) is referenced in FIGS. 6-10D. FIG. 6 shows the pump system (200) integrated into the lower casing (107), connected to the reservoir (203) for the fluid to be extracted and to the internal reservoir (215). FIG. 7 shows a cross-sectional side view of the pump system (200), revealing the fluidic circuit. FIG. 8 shows a side view of the pump body (201) from which the valve piston (202) emerges. FIG. 9 shows a radial cross-section of the pump system (200), with the pump body (201) cut in two axially offset planes to reveal the ports (212 and 213) simultaneously. FIGS. 10A-10D highlight the geometry of the three ports (211, 212, 213) of the pump body (201) and their advantage over those used in international patent application WO2018141697A1.

More particularly, and as shown in FIGS. 6 and 7, the pump is composed of a pump body (201) and two pistons, a pump piston (205) and a valve piston (202), defining between them an internal cavity (208) of variable volume. These two pistons are each connected to a rack (248, 258) that can be set in axial motion by a drive system (240, 250) comprising a mechanical motion reducer (241, 251), the input of which is coupled to an electric motor (242, 252). The fluid transfer within the internal cavity (208) is made possible by the cooperation of multiple ports (211, 212, 213) with an internal channel (209) of the valve piston (202) opening at one end into the internal cavity (208) and terminated by an opening (221) at its other end. The precise axial positioning of the valve piston (202) enables selective cooperation of one of the ports (211, 212, 213) with the pump opening (221).

In the example shown in FIG. 6, port (211) is connected to the reservoir (203) of the fluid to be extracted, for example, a vial, the port (212) is an outlet port and is connected to the injection system, the port (213) is connected to the internal reservoir (215) of the medical device. The port (211) and port (213) can be used alternatively as input or output ports. A pump outlet port is a port from which the fluid flows exclusively so as to exit the pump's internal cavity (208). In the initial state, the valve piston (202) and pump piston (205) are in face-to-face contact or are only slightly apart, so that the internal cavity (208) has a small volume of air.

Two injection sequences are preferred, without limiting the present disclosure; one uses the internal reservoir, and the other is direct injection.

The first injection sequence involves transferring the liquid from the vial to the internal reservoir, then transferring the liquid from the internal reservoir to the injection system. The first pumping sequence then consists of the following steps:
moving the valve piston (202) and the pump piston (205) synchronously, by means of their respective drive systems (240, 250), to align the pump port (221) with the port (211) connected to the reservoir (215),
moving the pump piston (205) alone, by means of the drive system (250), so as to increase the volume of the internal cavity (208) located between the valve piston (202) and the pump piston (205), thus creating negative pressure leading to the liquid being drawn from the reservoir (203) into the internal cavity (208) through the internal channel (209),
once the internal cavity is completely filled, moving the pistons (202, 205) synchronously, using their respective drive systems (240, 250), so as to align the pump port (221) with the port (213) connected to the internal reservoir (215),
translating the pump piston (205), alone, by means of its drive system (250), so as to reduce the volume of the internal cavity (208) in order to expel all the fluid contained in the internal cavity (208) through the internal channel (209) and toward the internal reservoir (215), and
once the liquid has been expelled, repeating the above steps successively until all the liquid contained in the reservoir (203), or a predetermined quantity, has been transferred to the internal reservoir (215).

Once the fluid has been completely transferred to the internal reservoir (215), injection into the patient can begin. The first step is therefore to trigger the needle insertion system, as described, for example, in international patent application WO2018141697A1. Once the canula has been inserted into the subcutaneous tissue, a new pumping sequence takes place in the following steps:
moving the valve piston (202) and the pump piston (205) synchronously, by means of their respective drive systems (240, 250), to align the pump port (221) with the port (213) connected to the internal reservoir (215),
moving the pump piston (205) alone, by means of the drive system (250), so as to increase the volume of the internal cavity (208) located between the valve piston (202) and the pump piston (205), thus creating negative pressure leading to the liquid being drawn from the internal reservoir (215) into the internal cavity (208) through the internal channel (209),
once the internal cavity is completely filled, moving the pistons (202, 205) synchronously, using their respective drive systems (240, 250), so as to align the pump port (221) with the port (212) connected to the injection system,
translating the pump piston (205), alone, by means of its drive system (250), so as to reduce the volume of the internal cavity (208) in order to expel all the fluid contained in the internal cavity (208) through the internal channel (209) and toward the injection system, and
once the liquid has been completely expelled, repeating the above steps successively until all the liquid contained in the internal reservoir (215), or a predetermined quantity, has been transferred to the injection system.

The second injection sequence eliminates the need to transfer fluid to the internal reservoir. The first step is therefore to trigger the needle insertion system, as described, for example, in international patent application WO2018141697A1. Once the canula has been inserted into the subcutaneous tissue, a pumping sequence takes place in the following steps:
moving the valve piston (202) and the pump piston (205) synchronously, by means of their respective drive systems (240, 250), to align the pump port (221) with the port (211) connected to the reservoir (203),
moving the pump piston (205) alone, by means of the drive system (250), so as to increase the volume of the internal cavity (208) located between the valve piston (202) and the pump piston (205), thus creating negative pressure leading to the liquid being drawn from the reservoir (203) into the internal cavity (208) through the internal channel (209),
once the internal cavity is completely filled, moving the pistons (202, 205) synchronously, using their respective drive systems (240, 250), so as to align the pump port (221) with the port (212) connected to the injection system,
translating the pump piston (205), alone, by means of its drive system (250), so as to reduce the volume of the internal cavity (208) in order to expel all the fluid contained in the internal cavity (208) through the internal channel (209) and toward the injection system, and
once the liquid has been completely expelled, repeating the above steps successively until all the liquid contained in the reservoir (203), or a predetermined quantity, has been transferred to the injection system.

These two injection sequences do not limit the present disclosure, and other alternatives may be envisaged. In particular, the pump system (200) can be used for drug reconstitution functions, for example, to reconstitute a lyophilized drug and a diluent. The internal reservoir (215) could then be a cartridge pre-filled with a diluent, and a pump sequence could be performed to transfer this diluent to a reservoir (203) containing a lyophilized drug in order to dissolve it. Once the lyophilized drug has dissolved, the transfer is carried out in reverse, from the reservoir (203) to the internal reservoir (215), for subsequent injection into the patient. It is also conceivable to carry out multiple successive transfers, partial or total, between the reservoir (203) and the internal reservoir, as this may facilitate dissolution of the lyophilized drug in the diluent.

Alternatively, the external reservoir (203) could be dispensed with altogether, the internal reservoir (215) being a cartridge pre-filled with the fluid to be injected. In this case, the fluid is transferred from the internal reservoir (215) to the internal cavity and then to the injection system. This sequence of steps is repeated as many times as necessary.

As can be seen in FIG. 8, the ports (211, 212, 213) are staggered, with the ports (211 and 213) lying in the same longitudinal plane, the port (212) being radially offset and lying axially between the two ports (211 and 213). This staggered configuration is particularly advantageous for limiting the stroke required by the valve piston (202) to select one of the ports (211, 212, 213), as this procedure must be repeated multiple times during an injection cycle. The axial proximity of the ports (211, 212, 213) has a direct influence on the durability of the battery (260) and therefore on the overall efficiency of the device. The downside is that the axial dimension of the ports is limited and therefore the axial positioning of the valve piston (202) must be more precise, as incorrect positioning results in a reduction in the cross-section of the fluid flow. Depending on the viscosity of the fluid, this can lead to significant pressure losses and therefore a reduction in fluid flow.

It is assumed from this principle that the positioning of the pump's valve piston opening opposite one of the ports is a critical point in the current prior art, as embodied in international patent application WO2018141697A1. Ensuring a minimum fluid flow cross-section in the event of axial piston “mispositioning” has the advantage of making the system more stable and robust, and limiting pressure losses, thus guaranteeing accurate flow, the quantity of medication injected into the patient, and injection time. The present disclosure proposes to overcome this problem by suggesting two improvements that can be used independently or in combination.

FIGS. 8 and 9 highlight one of these improvements. The staggered arrangement of the ports (211, 212, 213) results in a tangential misalignment of these holes with respect to the opening (221) of the valve piston (202). So as not to reduce pump flow, the port (211) of the valve piston (202) has a tangential flared section (223, 224) on either side of its radial end facing the ports (211, 212, 213). These flared sections are delimited by tangential beads (225, 226) and axial beads (227, 228) made of a flexible, deformable material, such as silicone or a soft thermoplastic, to match the cylindrical inner surface of the pump body (201) and ensure sealing. To this end, the valve piston has a hard core (230), wherein the internal channel (209) connected to the opening (221) is formed, this hard core (230) being overmolded by an elastomer (229) to form the tangential flared sections (223, 224). FIG. 9 shows a radial cross-section of the valve body, with the left and right halves of the valve not in the same axial plane, but in two half-planes at mid-port (212) and mid-port (213), respectively. This cross-section shows the correct correspondence between the tangential flared sections (223, 224) and the ports (212, 213) offset tangentially to the opening (221) of the valve piston (202).

The second improvement in valve piston position tolerance is shown in FIGS. 7, 8, and 10A-10D. In particular, the ports (211, 212, 213) of the pump body (201) are extended by conical cavities (231, 232, 233) for coupling with the cylindrical flexible tubes (261, 262, 263), visible in FIG. 4, connecting elements of the fluidic system, while ensuring a radial stop of these tubes thanks to a shoulder (220, 222). Consequently, the diameter of the ports (211, 212, 213) must be smaller than the outer diameter of the flexible tubing and ideally larger than the inner diameter of the flexible tubing, so as not to limit flow. Although the shoulder secures the mechanical connection, it is not necessary for it to be present around the entire circular periphery of the flexible tubing. In this way, the ports (211, 212, 213) can advantageously have an oblong periphery to increase the cross-section opposite the opening of the valve piston (202), while at the same time providing a mechanical stop for the flexible tubing.

The term “oblong periphery” means that the cross-section of the ports (211, 212, 213), as shown in FIG. 8, is not disc-shaped but has a different length in the axial and tangential directions. In the example shown in FIG. 8, and in FIGS. 10C and 10D, the cross-section is a groove in the tangential direction, terminated by two half-cylinders. However, the present disclosure is not limited to this type of shape, but includes any type of elongated section, such as a rectangle or ellipse. Additionally, the long length is not limited to the tangential direction, but can also be realized in the radial direction and will be chosen according to the arrangement of the ports (211, 212, 213).

FIGS. 10A-10D different scenarios for positioning the valve piston (202), with FIGS. 10A and 10B representing the case of a port (221) and a port (211) both with a disc section, as realized in patent application WO2018141697A1. FIG. 10A shows a cylindrical port (211) with a cross-section of 0.196 mm² for a diameter of 0.5 mm and being perfectly aligned with an identical port (221), the fluid flow cross-section is represented by cross-hatching. FIG. 10B shows the progress of the hatched passage cross-section when there is an axial misalignment between the opening (221) and the port (211). It may be noted that an offset of 0.1 mm results in a remaining cross-sectional area of 0.122 mm², that is, a reduction by over 35%. The oblong shape of the port (211) according to the present disclosure is shown in FIGS. 10C and 10D, and offers a fluid passage cross-section of 0.321 mm² in the same axial space when combined with an identical opening or a disc opening with a tangential flared section as shown. FIG. 10D shows the evolution of the hatched passage cross-section in the event of incorrect axial positioning of the pump. Note that for an offset of 0.1 mm, the cross-section decreases to leave a passage of 0.268 mm². Note also that up to an axial offset of 0.2 mm, that is, a passage cross-section of 0.198 mm², the passage cross-section remains greater than or equal to that obtained for a disc section port of the same axial dimensions. This means that even with an offset of 0.2 mm, corresponding to a limit tolerance, the shape of the invention ensures a fluid flow cross-section equivalent to the cylindrical shape of a 00.5 mm as achieved in the prior art. The form of the invention therefore enables precise positioning at ±0.2 mm without impacting the pressure losses in the system.

It should be noted that the present disclosure is not limited to an oblong shape, but can be extended to any shape that increases the cross-sectional area for fluid flow, while ensuring radial shut-off of the tubing connected to the pump body.

### Detailed Description of Activation by Vial Insertion

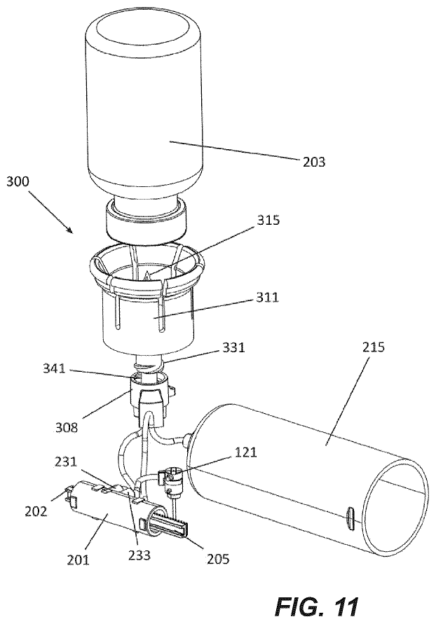

The medical device according to a variant of the present disclosure, to be appreciated through FIGS. 11-20, is suitable for interfacing with an external reservoir (203), such as a vial. To simplify the use of the device and its reliability, the vial must be coupled to an adapter (311) designed to fit into a dock (308) of the medical device, having complementary shapes. In the example shown in FIG. 11, the adapter (311) has a hollow needle (315) for piercing the septum of the vial (312) and withdrawing the fluid contained therein, the hollow needle (315) being connected to a central channel of the adapter (311). This operation is carried out before the medical device is activated, and the configuration of the septum and needle are such that only fluid flow through the internal channel is possible, thus preventing air from entering the vial (312) and thus preventing spontaneous flow of the fluid contained in the vial. The medical device is activated by inserting the adapter (311) into the dock (308). To this end, the adapter (311) and the dock (308) can be screwed into each other so that complementary central tubes (320, 321) on both parts cooperate to provide a sealed flow channel for conveying fluid between the vial and the medical device. In the example shown, the central tubes (320, 321) cooperate by means of a conical fitting to form a Luer connection, but the present disclosure extends to any type of connection compatible with medical applications. When the adapter (311) is screwed into the receiving piece (308), the torque exerted by the adapter (311) on the receiving piece (308) drives the latter in rotation in order to activate the electronic system (310) and authorize transfer of the drug to the internal reservoir as described in international patent application WO2018141697A1. Once the medication has been transferred, the user can disconnect the adapter (311) by unscrewing it, this movement then returning the dock (308) to its initial position and simultaneously deactivating the electronic system (310). During the sequence of activation and deactivation of the system by the means described, the user can be informed about the status of the medical device by haptic or visual signals, such as vibrations or the powering on of LEDs. The triggering of the signals can be directly activated by the user's actions on the device.

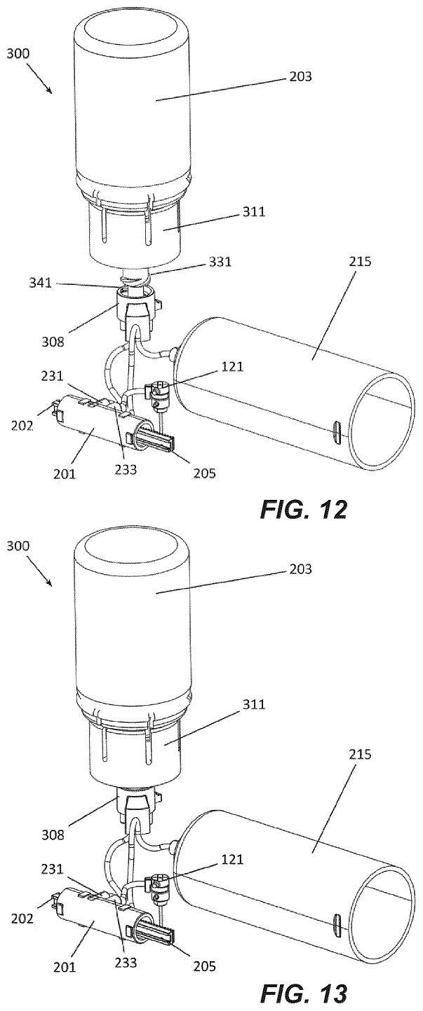

FIGS. 11-13 show the isolated fluidic system (300) of one embodiment of the present disclosure. FIGS. 11 and 13 are perspective views showing the various steps of connecting an external reservoir (203), with FIG. 11 showing an exploded view of the various unconnected elements, FIG. 12 showing the connection of the reservoir (203) to the adapter (311) and FIG. 13 showing the connection of the reservoir (203) and adapter (311) assembly to the dock (308). These various figures also show the other components of the fluidic system, that is, the fluidic part of the pump system (200), the internal reservoir (215) and the needle holder (121) of the injection system, these various elements being connected by flexible tubes, one end of which is inserted into cavities (231, 233) in the pump body (201). Fluid is directed to either element by the movements of the valve piston (202) and pump piston (205) described in this disclosure. In particular, FIG. 11 shows the presence of a hollow needle (315), designed to pierce the septum of the external reservoir to make a sealed connection between the external reservoir (203) and the fluidic system piping (300). The assembly shown in FIG. 13 is obtained, from FIG. 12, by screwing together the adapter (311) and the dock (308) via a thread (331) and an internal thread (330).

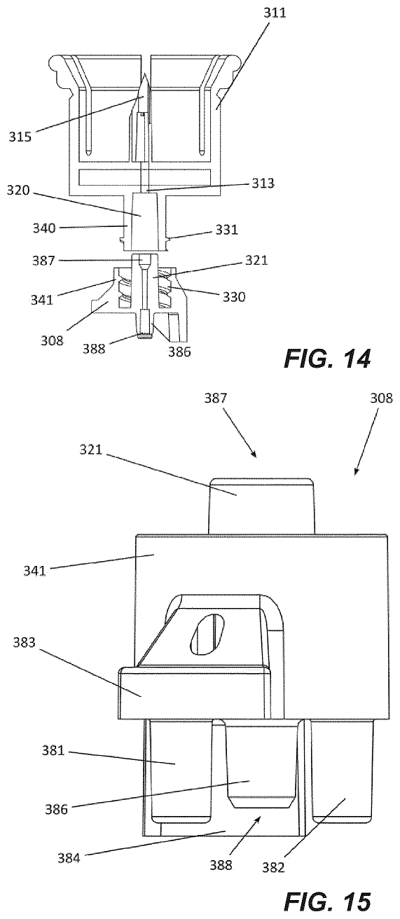

FIG. 14 shows a sectional view of the adapter (311) and dock (308) before assembly, to better visualize the fluidic connection between these parts and how they connect. In particular, complementary central manifolds (320, 321) belonging respectively to the adapter (311) and to the docking part (308) ensure connection of the fluidic outlet port (313) of the adapter (311) to the inlet port (387) of the dock (308). The hollow needle (315) of the adapter (311) has an internal channel terminating in the outlet port (313) that opens into the central tube (320). The dock (308) is formed by a cylindrical body (341) having an axially open cavity wherein the central tube (321) extends, the inner cylindrical surface of the cavity having the thread (330) cooperating with the thread (331) of the adapter (311). At its other axial end, the dock (308) has a hollow axial protuberance (386) with a slightly conical periphery so that it can easily be plugged into a flexible tube, to ensure that fluid is transferred to the pump body. In this way, a conduit passes axially through the dock, opening on the one hand into the inlet port (387) at one axial end of the central tube (321), and on the other hand into an outlet port (388) located at the end of the axial protuberance (386).

The central tubes (320, 321) are advantageously conical in shape, known as a “luer taper,” enabling tight coupling in compliance with ISO 80369, when sufficient contact force is ensured. The interlocking of the complementary central tubes (320, 321) and the application of the force required for sealing are ensured by the cooperation of a thread (331) complementary to a female thread (330), respectively made in the hollow cylindrical protuberance (340) of the adapter (311) and in the cylindrical body (341) of the dock (308), surrounding the central tubes (320, 321). Thus, once the adapter (311) and dock (308) are assembled, any fluid entering through the hollow needle (315) is directed to the outlet port (388) without further leakage.

It should be noted that the arrangement of this connection does not limit the present disclosure, as the person skilled in the art can easily imagine alternatives for tubes of two complementary parts in a watertight manner by a screwing method.

FIG. 15 gives a more detailed view of the dock (308) shown in FIGS. 11-14. The upper part of the figure shows the central tube (321) emerging from the hollow cylindrical body (341), which has a thread for screwing onto the adapter. Also shown is the cylindrical body (341) for guiding the rotation of the dock (308) by cooperation with an annular protrusion of the lower casing (107) of the housing. Finally, in order to ensure that rotation stops once the adapter has been fully screwed into the dock (308) by the user, two cylindrical axial protuberances (381, 382) extend from the cylindrical body (341), these axial protuberances (381, 382) are located radially on either side of the axial protuberance (386) and are designed to extend into the lower casing (107) of the housing, so as to cooperate with an elastically deformable part, to ensure that the final angular position of the dock is maintained. The final angular position is obtained by abutment of a radial protuberance (383), radially extending from the lower axial end of the cylindrical body (341), against a complementary abutment of the lower casing of the housing. Finally, a partial annular protuberance (384) may be noted, axially extending the cylindrical body (341) over a limited angular portion. This partial annular protuberance (384) is called an activation arm and enables the medical device activation switch to be engaged or released depending on the angular position of the dock (308). The partial annular protuberance (384) also has a cylindrical inner surface concentric with a cylindrical protrusion of the lower casing of the housing, in order to ensure the rotational guidance of the dock (308).

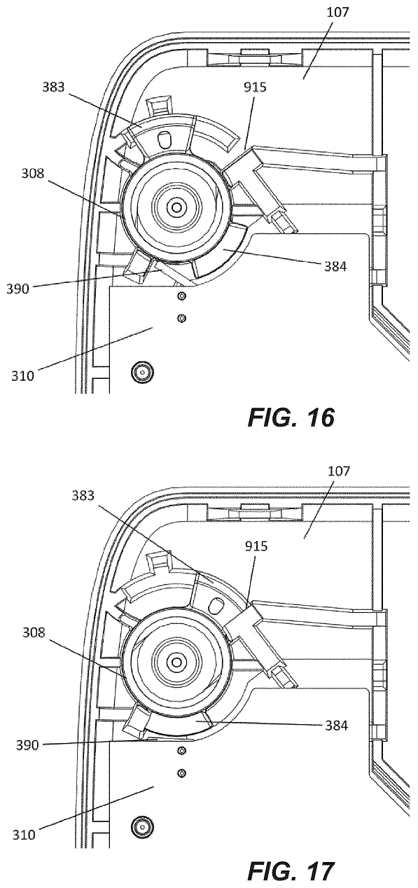

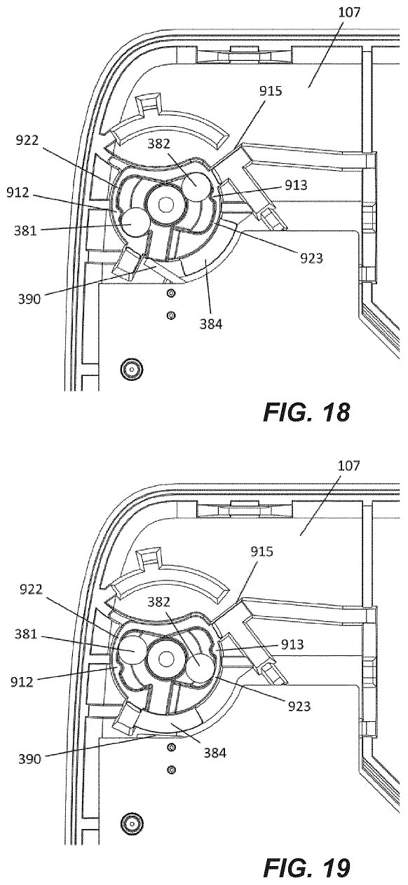

FIGS. 16 and 18 show the dock (308) installed in the lower casing (9) of the housing in its initial state, that is, before insertion of the vial fitted with its adapter. FIG. 16 shows a view from an axial angle and FIG. 18 shows a cross-sectional view, at the level of the axial protuberances (381, 382), from the same angle. When screwed in, the adapter is inserted axially into the dock (308), with the dock remaining stationary, until the central tubes are in coaxial contact so as to generate wedging. Therefore, if additional torque is applied to the adapter, this torque is transmitted to the dock (308), whose rotation is stopped by the axial protrusions (381, 382) bearing against the beads (912, 913) of the lower casing (107) of the housing. The beads (912, 913) are protrusions located in flexible beams (922, 923) that can be elastically deformed by the axial protrusions (381, 382), when a threshold torque is applied, enabling the dock to rotate until the radial protuberance (383), together with the partial annular protuberance (384), abuts against complementary stops (915, 916) on the lower casing (107) of the housing. This final position is shown in FIGS. 17 and 19, corresponding to FIGS. 16 and 18, respectively. It may be noted that the axial protrusions (381, 382) can be deformed alternatively or in addition to the flexible beams (922, 923) to allow rotation of the dock (308) when a torque greater than the threshold torque is applied thereto.

As the docking station (308) rotates, the partial annular protuberance (384) presses against the lever of the medical device activation switch (390) on the electronic system (310). At the end of movement, the radial protuberance (383) and the partial annular protuberance (384), when they come into contact with the respective complementary stops (915, 916), absorb any excess torque supplied by the user on the dock (308), preventing this excess torque from being reflected on the axial protuberances (381, 382), thus avoiding damage thereto.

It should be noted that the deformation torque required to rotate the dock (308) is greater than the torque required to guarantee the tightness of the connection according to ISO 80369. This deformation serves as haptic feedback to the user to indicate successful vial installation and activation of the medical device.

It should also be noted that the final angular position is not irreversible. If the user applies a torque of the same amplitude as that deployed to activate the device, but in the opposite direction, the flexible beams (922, 923) can again be elastically deformed by the axial protrusions (381, 382), allowing the dock to rotate in the opposite direction to the initial state shown in FIGS. 16 and 18. This operation is accompanied by the disarming of the switch (390), indicating to the medical device that the vial is no longer engaged. The user can continue to unscrew the adapter (311) to remove the vial (312).

This activation system offers a function that guarantees the user a monitored and secure vial insertion to limit any risk of misuse.

1. A device for transdermal drug administration comprising;
a housing having a lower casing defining a cutaneous connection surface with an opening for the passage of a transdermal injection canula;
a reservoir containing an active ingredient intended to be administered;
a pump system comprising a pump body, a valve piston and/or a pump piston that can be axially moved in the pump body by at least one drive system; and
a transdermal injection system and an electronic control system for operating the drive system(s) of the pump system and for releasing the transdermal injection system;
wherein the injection system is controlled by a translational element interacting with a connection means inserted between the pump piston or the motorized valve piston on one side, and the injection system on the other side, the connection means being movable between a first position that the connection means occupies until the injection system is activated, and a second, irreversible position wherein the connection means is switched by the triggering of the injection system, and in which second position the connection means is retracted so as to no longer present any zone of possible interaction with the piston, over an entire stroke of the piston.

2. The device of claim 1, wherein the electronic system controls:
over a limited part of a nominal axial stroke of the pump piston or the valve piston, a pumping function or a valve function only, and
over at least an additional part of the stroke, the axial displacement of the pump piston or the valve piston so as to drive an additional function independent of the pumping function or the valve function, the additional function not being activated during the axial displacement of the pump piston or the valve piston over the nominal stroke.

3. The device of claim 2, wherein the additional function is the triggering of the transdermal injection system.

4. The device of claim 3, wherein the transdermal injection system controls:
a mechanism for actuating an assembly comprising a needle connected to a needle holder, the canula connected to a canula holder, and a guide element configured to guide the axial displacement of the needle and canula;
the actuating mechanism comprising:
a cam element rotatable relative to the needle guide element, the needle holder and canula holder each comprising a protuberance cooperating with a rib of the guide element, and
a pre-loaded spring for imparting rotational movement to the cam element relative to the needle guide element, for sequentially controlling needle insertion and needle retraction movement such that the needle and canula are moved from a retracted position to an extended position upon rotation of the cam element through a first predetermined angle, by cooperation of the protuberances of the needle holder and canula holder with the rib of the cam element, and such that the needle is returned to the retracted position upon further rotation of the cam element through a second predetermined angle by cooperation of the protuberance of the needle holder with the rib of the cam element, whereby during rotation through the second predetermined angle, the protuberance of the canula holder is maintained in the extended position by cooperation with the cam element.

5. The device claim 1, wherein the injection system includes a locking device bearing against a wall of the cam element so as to prevent rotation of the cam element subjected to the action of the pre-loaded spring.

6. The device of claim 5, wherein the locking device is provided with a connection means rotatable about an axis and a translational element translatable in a plane orthogonal to the axis, a distal end of the translational element being in contact with the wall of the cam element, a proximal end resting on a surface of the connection means, an opposite surface of the connection means resting against a surface of a protuberance of the lower casing impeding any translational movement of the translational element, and wherein the end of the rod of one of the pistons is movable through the additional stroke to a position where the end of the rod comes into contact with the connection means to impart a rotational movement thereto, the rotational movement causing the release of the locking device by releasing the contact between the proximal end of the translational element and the surface of the connection means.

7. The device of claim 2, wherein the additional function is the triggering of a system for perforating a membrane separating the recess for a cartridge containing an active ingredient from the interior space of the housing.
